(19) United States
(12) Patent Application Publication
(10) Pub. No.: US 2024/0122520 A1
(43) Pub. Date: Apr. 18, 2024

(54) DEFIBRILLATOR FOR ASSESSING A LIKELIHOOD OF PATIENT REFIBRILLATION

(71) Applicant: HeartSine Technologies Limited, Belfast (GB)

(72) Inventors: Dymphna DONAGHY, Co. Donegal (IE); Johnny ANDERSON, Holywood (GB); Adam HARVEY, Hillsborough (GB); Amy KERNAGHAN, Dundonald (GB); Olibhear McALISTER, Belfast (GB)

(21) Appl. No.: 18/378,774

(22) Filed: Oct. 11, 2023

(30) Foreign Application Priority Data
Oct. 12, 2022 (EP) 22201061.3

(51) Int. Cl. A61B 5/33; A61B 5/361; A61N 1/39

(52) U.S. Cl. CPC A61B 5/33; A61B 5/361; A61N 1/39044; A61N 1/3925

(57) ABSTRACT

A device may include an electrocardiogram (ECG) signal input configured to receive ECG signals of a patient. A device may include an ECG analysis system, in communication with the ECG signal input, configured to receive patient ECG signals, analyse the patient ECG signals to generate an ECG refibrillation indicator representing a likelihood of refibrillation of the patient and use the ECG refibrillation indicator to choose treatment for the patient.

The present application claims priority to EP 22201061.3, filed Oct. 12, 2022, the content of which is incorporated herein in its entirety.

### FIELD OF THE INVENTION

The present disclosure relates to a defibrillator, particularly but not exclusively, an automated external defibrillator (AED), for assessing a likelihood of refibrillation of a patient and using this to choose a treatment for the patient.

### BACKGROUND

Current AEDs deliver defibrillation shock therapy to patients presenting with a shockable arrythmia, ventricular fibrillation (VF), which is measured using electrocardiogram (ECG) signals of the patient. The defibrillation shock therapy is designed to depolarize the entirety of the myocardium of the patient's heart, remove the arrythmia and restore the coordinated electrical activity of the heart to a normal, or sinus, rhythm. The vast majority of defibrillation shock therapy is successful at terminating VF. However, refibrillation of a patient's heart can occur, where the ventricular fibrillation (VF) arrythmia returns.

In addition to the delivery of defibrillation shock therapy to the patient, a defibrillator often instructs cardiopulmonary resuscitation (CPR) to be administered to a patient by a user of the defibrillator. CPR compensates for the inability of the patient's heart to pump blood around the body whilst experiencing a VF arrythmia. The time at which CPR is administered has been shown to be important in the treatment of the patient and attaining and maintaining a normal sinus heart rhythm after defibrillation shock therapy.

### BRIEF SUMMARY

This present disclosure addresses weaknesses in defibrillators outlined above. According to one aspect of the invention there is provided a defibrillator for assessing a likelihood of patient refibrillation, including: an electrocardiogram (ECG) signal input configured to receive ECG signals of the patient, an ECG analysis system, in communication with the ECG signal input, configured to receive the patient ECG signals, analyse the patient ECG signals to generate an ECG refibrillation indicator representing a likelihood of refibrillation of the patient and use the ECG refibrillation indicator to choose treatment for the patient.

In some aspects, the techniques described herein relate to a defibrillator for assessing a likelihood of patient refibrillation, including: an electrocardiogram (ECG) signal input configured to receive ECG signals of a patient; and an ECG analysis system, in communication with the ECG signal input, configured to receive patient ECG signals, analyse the patient ECG signals to generate an ECG refibrillation indicator representing a likelihood of refibrillation of the patient and use the ECG refibrillation indicator to choose treatment for the patient.

In some aspects, the techniques described herein relate to a method including: receiving patient ECG signals; analyzing the patient ECG signals for shockable arrythmia; determining the shockable arrythmia and preparing to deliver a defibrillation shock; before the defibrillation shock, analyzing the patient ECG signals and generating an ECG pre-shock refibrillation indicator used to choose a patient treatment; delivering the defibrillation shock; after the defibrillation shock, analysing the patient ECG signals and generating an ECG post-shock refibrillation indicator, combining the ECG post-shock refibrillation indicator with an ECG pre-shock refibrillation indicator to generate an aggregate ECG refibrillation indicator; using the aggregate ECG refibrillation indicator to choose the patient treatment; after the defibrillation shock, analysing patient impedance signals, generating an impedance post-shock refibrillation indicator, combing the impedance post-shock refibrillation indicator with the aggregate ECG refibrillation indicator to generate an aggregate ECG/impedance refibrillation indicator; using the aggregate ECG/impedance refibrillation indicator to choose the patient treatment; and repeating the analyzing of the patient ECG signals and the analyzing of the patient impedance signals and using an updated aggregated ECG/impedance refibrillation indicator to choose the patient treatment.

In some aspects, the techniques described herein relate to a device including: a processor; a computer-readable memory storing instructions which, when executed by the processor, cause the processor to perform operations including: receiving patient ECG signals; analyzing the patient ECG signals for shockable arrythmia; determining the shockable arrythmia and preparing to deliver a defibrillation shock; before the defibrillation shock, analyzing the patient ECG signals and generating an ECG pre-shock refibrillation indicator used to choose a patient treatment; delivering the defibrillation shock; after the defibrillation shock, analysing the patient ECG signals and generating an ECG post-shock refibrillation indicator, combining the ECG post-shock refibrillation indicator with an ECG pre-shock refibrillation indicator to generate an aggregate ECG refibrillation indicator; using the aggregate ECG refibrillation indicator to choose the patient treatment; after the defibrillation shock, analysing patient impedance signals, generating an impedance post-shock refibrillation indicator, combing the impedance post-shock refibrillation indicator with the aggregate ECG refibrillation indicator to generate an aggregate ECG/impedance refibrillation indicator; using the aggregate ECG/impedance refibrillation indicator to choose the patient treatment; and repeating the analyzing of the patient ECG signals and the analyzing of the patient impedance signals and using an updated aggregated ECG/impedance refibrillation indicator to choose the patient treatment.

### BRIEF DESCRIPTION OF THE SEVERAL VIEWS OF THE DRAWINGS

Illustrative aspects of the present application are described in detail below with reference to the following figures:

FIG. 1 illustrates a defibrillator;

FIG. 2 illustrates aa method aspect of this disclosure; and

FIG. 3 illustrates a computing device in according with some aspects of this disclosure.

### DETAILED DESCRIPTION

What is needed in the art is an improved ECG system. The ECG analysis system disclosed herein can analyse the patient ECG signals before a defibrillation shock and generate a ECG pre-shock refibrillation indicator. The defibrillator may use the ECG pre-shock refibrillation indicator to choose treatment for the patient. The defibrillator may use the ECG pre-shock refibrillation indicator to choose to deliver defibrillation shock treatment to the patient at an energy equal to a default energy or deliver defibrillation shock treatment to the patient at an energy greater than the default energy.

The ECG analysis system may analyse the patient ECG signals after delivery of a defibrillation shock and generate at least one ECG post-shock refibrillation indicator. The defibrillator may combine the ECG pre-shock refibrillation indicator and the at least one ECG post-shock refibrillation indicator to generate an aggregate ECG refibrillation indicator. The defibrillator may use the aggregate ECG refibrillation indicator to choose treatment for the patient. The defibrillator may use the aggregate ECG refibrillation indicator to choose to start CPR treatment of the patient or delay CPR treatment of the patient. The defibrillator may issue a start CPR signal or issue a delay CPR signal. The defibrillator may comprise a user interface configured to receive the start CPR signal and deliver a start CPR instruction to a user and to receive the delay CPR signal and deliver a delay CPR instruction to the user.

For a patient experiencing a VF arrythmia, after receiving a defibrillation shock, an ECG signal from the patient may have a waveform exhibiting anything from a continuation of the VF arrythmia, through an irregular partial sinus rhythm, to a regular sinus rhythm. When the ECG signal has a waveform comprising an irregular partial sinus rhythm, the patient's heart will not achieve proper perfusion of blood around the circulatory system. Even when the ECG signal has a waveform comprising a regular sinus rhythm, the patient's heart may not achieve proper perfusion. In these circumstances, CPR is often instructed, to compensate for lack of perfusion. The timing of administration of CPR to a patient influences the likelihood of refibrillation of the patient's heart. Different patients will benefit from shorter or longer delay periods before commencement of CPR. The CPR delay period can be tailored for a patient, based on the expectation of refibrillation of that patient, to reduce the likelihood of refibrillation and the requirement for further defibrillation shock therapy.

Before delivery of a defibrillation shock, the ECG analysis system may take one or more samples of the ECG signals over one or more periods of time. Each period of time may be in the range of 1 sec to 10 sec. When two or more samples are taken, the periods of time may be any of adjacent periods of time, spaced periods of time.

The ECG analysis system may analyse one or more samples of the ECG signals to detect one or more parameters of the or each sample and may use the or each or at least some of the parameters as one or more inputs of a refibrillation indicator model which generates an ECG pre-shock refibrillation indicator.

The ECG analysis system may analyse one or more whole samples of the ECG signals to detect one or more parameters of the or each whole sample. The ECG analysis system may use the or each or at least some of the parameters as one or more inputs of a refibrillation indicator model which generates an ECG pre-shock refibrillation indicator. The ECG analysis system may aggregate one or more equivalent parameters of multiple whole samples. Aggregation of equivalent parameters may comprise averaging the equivalent parameters. The ECG analysis system may use the or each or at least some of the aggregate parameters as one or more inputs of a refibrillation indicator model which generates an ECG pre-shock refibrillation indicator.

The ECG analysis system may analyse one or more samples of the ECG signals to detect one or more parameters at one or more sample instances of the or each sample. The ECG analysis system may use the or each or at least some of the parameters as one or more inputs of a refibrillation indicator model which generates an ECG pre-shock refibrillation indicator. The ECG analysis system may aggregate one or more equivalent parameters of the one or more samples. Aggregation of equivalent parameters may comprise averaging the equivalent parameters. The ECG analysis system may use the or each or at least some of the aggregate parameters as one or more inputs of a refibrillation indicator model which generates an ECG pre-shock refibrillation indicator.

The one or more parameters may be any of time domain parameters, frequency domain parameters, wavelet parameters. The one or more parameters may be any of an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum amplitude spectrum area (AMSA), an ECG signal sample minimum AMSA, an ECG signal sample centroid frequency, an ECG signal sample power. Before delivery of a defibrillation shock, the ECG analysis system may receive ECG signals having a VF waveform, i.e. exhibiting a VF arrythmia. The one or more parameters may be parameters of a VF waveform.

Each of the parameters will correlate with a prospect of success of defibrillation treatment and a likelihood of refibrillation of the patient.

The refibrillation indicator model may weight the or each input according to a strength of indication of refibrillation. The strengths of indication of refibrillation of the inputs may be determined from research. The refibrillation indicator model may use the or each weighted input to generate an ECG pre-shock refibrillation indicator.

For multiple samples, the refibrillation indicator model may generate an ECG pre-shock refibrillation indicator for each sample. For multiple samples, the refibrillation indicator model may aggregate the ECG pre-shock refibrillation indicators to generate an ECG pre-shock refibrillation indicator. For multiple samples, the refibrillation indicator model may weight the ECG pre-shock refibrillation indicators and then aggregate the weighted ECG pre-shock refibrillation indicators to generate an ECG pre-shock refibrillation indicator.

The refibrillation indicator model may compare the ECG pre-shock refibrillation indicator to at least one pre-determined ECG pre-shock refibrillation threshold and generate a flag which prompts the defibrillator to deliver a treatment to the patient. The at least one pre-determined ECG pre-shock refibrillation threshold may be generated using data from patients who have received defibrillation shock therapy. The at least one pre-determined ECG pre-shock refibrillation threshold may be generated using data from the patient receiving defibrillation shock therapy. When the ECG pre-shock refibrillation indicator is less than or equal to the pre-determined ECG pre-shock refibrillation threshold, the refibrillation indicator model may generate a flag which prompts the defibrillator to deliver defibrillation shock treatment to the patient at an energy equal to a default energy. When the ECG pre-shock refibrillation indicator is greater than the pre-determined ECG pre-shock refibrillation threshold, the refibrillation indicator model may generate a flag which prompts the defibrillator to deliver defibrillation shock treatment to the patient at an energy greater than the default energy.

The refibrillation indicator model may be a binary logistic regression model. The regression model may determine a likelihood of refibrillation using the one or more inputs of the model.

After delivery of a defibrillation shock, the ECG analysis system may take one or more samples of the ECG signals over one or more periods of time. The or each period of time may be in the range of 1 sec to 10 sec. When two or more samples are taken, the periods of time may be any of adjacent periods of time, spaced periods of time. The period of time may comprise a moving window of time.

The ECG analysis system may analyse one or more samples of the ECG signals to detect one or more parameters of the or each sample and may use the or each or at least some of the parameters as one or more inputs of a refibrillation indicator model which generates an ECG post-shock refibrillation indicator.

The ECG analysis system may analyse one or more whole samples of the ECG signals to detect one or more parameters of the or each whole sample. The ECG analysis system may use the or each or at least some of the parameters as one or more inputs of a refibrillation indicator model which generates an ECG post-shock refibrillation indicator. The ECG analysis system may aggregate one or more equivalent parameters of multiple whole samples. Aggregation of equivalent parameters may comprise averaging the equivalent parameters. The ECG analysis system may use the or each or at least some of the aggregate parameters as one or more inputs of a refibrillation indicator model which generates an ECG post-shock refibrillation indicator.

The ECG analysis system may analyse one or more samples of the ECG signals to detect one or more parameters at one or more sample instances of the or each sample. The ECG analysis system may use the or each or at least some of the parameters as one or more inputs of a refibrillation indicator model which generates an ECG post-shock refibrillation indicator. The ECG analysis system may aggregate one or more equivalent parameters of the one or more samples. Aggregation of equivalent parameters may comprise averaging the equivalent parameters. The ECG analysis system may use the or each or at least some of the aggregate parameters as one or more inputs of a refibrillation indicator model which generates an ECG post-shock refibrillation indicator.

The one or more parameters may be any of time domain parameters, frequency domain parameters, wavelet parameters. The one or more parameters may be any of an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum AMSA, an ECG signal sample minimum AMSA, an ECG signal sample centroid frequency, an ECG signal sample power.

After delivery of a defibrillation shock, the ECG analysis system may receive ECG signals having a waveform exhibiting one or more PQRST (provocation, quality, region, severity and timing) complexes. The one or more parameters may comprise any of an amplitude of a P wave of a PQRST complex, an amplitude of a QRS wave of a PQRST complex, variation of amplitudes of QRS waves of two or more PQRST complexes, an amplitude of a T wave of a PQRST complex, a width of a P wave of a PQRST complex, a width of a QRS wave of a PQRST complex, a width of a T wave of a PQRST complex, a ST segment of a PQRST complex, a coupling interval between a QRS complex and a premature, abnormal complex, an R-R interval between two or more PQRST complexes, a ST deviation, presence of one or more premature ventricular contractions.

Each of the parameters will correlate with a prospect of success of defibrillation treatment and a likelihood of refibrillation of the patient.

The refibrillation indicator model may weight the or each input according to a strength of indication of refibrillation. The strengths of indication of refibrillation of the inputs may be determined from research. The refibrillation indicator model may use the or each weighted input to generate an ECG post-shock refibrillation indicator.

For multiple samples, the refibrillation indicator model may generate an ECG post-shock refibrillation indicator for each sample. For multiple samples, the refibrillation indicator model may aggregate the ECG post-shock refibrillation indicators to generate an ECG post-shock refibrillation indicator. For multiple samples, the refibrillation indicator model may weight the ECG post-shock refibrillation indicators and then aggregate the weighted ECG post-shock refibrillation indicators to generate an ECG post-shock refibrillation indicator.

The refibrillation indicator model may be a binary logistic regression model. The regression model may determine a likelihood of refibrillation using the one or more inputs of the model.

The refibrillation indicator model may combine the ECG pre-shock refibrillation indicator and the ECG post-shock refibrillation indicator to generate an aggregate ECG refibrillation indicator. The refibrillation indicator model may combine the ECG pre-shock refibrillation indicator and the ECG post-shock refibrillation indicator by any of summing the ECG pre-shock refibrillation indicator and the ECG post-shock refibrillation indicator, taking a product of the ECG pre-shock refibrillation indicator and the ECG post-shock refibrillation indicator, taking a ratio of the ECG pre-shock refibrillation indicator to the ECG post-shock refibrillation indicator, using a model to combine the ECG pre-shock refibrillation indicator and the ECG post-shock refibrillation indicator.

The refibrillation indicator model may weight the ECG pre-shock refibrillation indicator and may weight the ECG post-shock refibrillation indicator. The refibrillation indicator model may combine the weighted ECG pre-shock refibrillation indicator and the weighted ECG post-shock refibrillation indicator to generate an aggregate ECG refibrillation indicator. The refibrillation indicator model may combine the weighted ECG pre-shock refibrillation indicator and the weighted ECG post-shock refibrillation indicator by any of summing the weighted ECG pre-shock refibrillation indicator and the weighted ECG post-shock refibrillation indicator, taking a product of the weighted ECG pre-shock refibrillation indicator and the weighted ECG post-shock refibrillation indicator, taking a ratio of the weighted ECG pre-shock refibrillation indicator to the weighted ECG post-shock refibrillation indicator, using a model to combine the weighted ECG pre-shock refibrillation indicator and the weighted ECG post-shock refibrillation indicator.

The refibrillation indicator model may compare the aggregate ECG refibrillation indicator with at least one pre-determined aggregate ECG refibrillation threshold and generate a flag which prompts the defibrillator to deliver a treatment to the patient. The at least one pre-determined aggregate ECG refibrillation threshold may be generated using data from patients who have received defibrillation shock therapy. The at least one pre-determined aggregate ECG refibrillation threshold may be generated using data from the patient receiving defibrillation shock therapy. When the aggregate ECG refibrillation indicator is less than or equal to the at least one pre-determined ECG refibrillation threshold, the refibrillation indicator model may generate a flag which prompts the defibrillator to start CPR treatment of the patient. The defibrillator may issue a start CPR signal. The user interface of the defibrillator may receive the start CPR signal and deliver a start CPR instruction to a user. When the aggregate ECG refibrillation indicator is greater than the at least one pre-determined ECG refibrillation threshold, the refibrillation indicator model may generate a flag which prompts the defibrillator to delay CPR treatment of the patient. The defibrillator may issue a delay CPR signal. The user interface of the defibrillator may receive the delay CPR signal and deliver a delay CPR instruction to the user.

When the aggregate or weighted aggregate ECG refibrillation indicator, which represents a likelihood of refibrillation of the patient, is low, the patient has a lower likelihood of refibrillation and immediate CPR can be commenced. When the aggregate or weighted aggregate ECG refibrillation indicator is high, the patient has a higher likelihood of refibrillation and delayed CPR is recommended.

At one or more further times after delivery of the defibrillation shock, the ECG analysis system may repeat analysis of the patient ECG signal and the refibrillation indicator model may generate a further ECG post-shock refibrillation indicator. The refibrillation indicator model may combine the ECG pre-shock refibrillation indicator and the further ECG post-shock refibrillation indicator to generate an aggregate ECG refibrillation indicator.

The refibrillation indicator model may compare the aggregate ECG refibrillation indicator with at least one pre-determined aggregate ECG refibrillation threshold and generate a flag which prompts the defibrillator to deliver a treatment to the patient. The at least one pre-determined aggregate ECG refibrillation threshold may be generated using data from patients who have received defibrillation shock therapy. The at least one pre-determined aggregate ECG refibrillation threshold may be generated using data from the patient receiving defibrillation shock therapy. When the aggregate ECG refibrillation indicator is less than or equal to the at least one pre-determined ECG refibrillation threshold, the refibrillation indicator model may generate a flag which prompts the defibrillator to start CPR treatment of the patient. The defibrillator may issue a start CPR signal. The user interface of the defibrillator may receive the start CPR signal and deliver a start CPR instruction to a user. When the aggregate ECG refibrillation indicator is greater than the at least one pre-determined ECG refibrillation threshold, the refibrillation indicator model may generate a flag which prompts the defibrillator to delay CPR treatment of the patient. The defibrillator may issue a delay CPR signal. The user interface of the defibrillator may receive the delay CPR signal and deliver a delay CPR instruction to the user.

The ECG analysis system may analyse the one or more samples of the ECG signal by operating one or more ECG signal analysis algorithms. The ECG analysis system may operate the one or more ECG signal analysis algorithms by executing software instructions of the or each algorithm. The ECG analysis system may comprise at least one ECG analysis processor configured to operate the one or more ECG signal analysis algorithms by executing software instructions of the or each algorithm.

The defibrillator may further comprise an impedance signal input configured to receive impedance signals of the patient and an impedance analysis system, in communication with the impedance signal input, configured to receive the patient impedance signal, analyse the patient impedance signal to generate at least one impedance refibrillation indicator representing a likelihood of refibrillation of the patient. The defibrillator may use the at least one impedance refibrillation indicator to choose treatment for the patient. The defibrillator may use the impedance refibrillation indicator to choose to start CPR treatment of the patient or delay CPR treatment of the patient. The defibrillator may issue a start CPR signal or issue a delay CPR signal. The defibrillator may comprise a user interface configured to receive the start CPR signal and deliver a start CPR instruction to a user and to receive the delay CPR signal and deliver a delay CPR instruction to the user.

The refibrillation indicator model may combine the aggregate ECG refibrillation indicator and the at least one impedance refibrillation indicator to generate an aggregate ECG/impedance refibrillation indicator. The refibrillation indicator model may compare the aggregate ECG/impedance refibrillation indicator with an aggregate ECG/impedance refibrillation threshold and generate a flag which prompts the defibrillator to deliver a treatment to the patient. When the aggregate ECG/impedance refibrillation indicator is less than or equal to the aggregate ECG/impedance refibrillation threshold, the refibrillation indicator model may generate a flag which prompts the defibrillator to commence CPR. The defibrillator may issue a commence CPR signal. When the aggregate ECG/impedance refibrillation indicator is greater than the aggregate ECG/impedance refibrillation threshold, the refibrillation indicator model may generate a flag which prompts the defibrillator to delay CPR. The defibrillator may issue a delay CPR signal.

After delivery of a defibrillation shock, the impedance analysis system may analyse the patient impedance signal to detect one or more impedance waveforms indicating one or more contractions of the patient's heart. The impedance analysis system may analyse the patient impedance signals to detect the one or more impedance waveforms indicating one or more contractions of the patient's heart by searching for the one or more impedance waveforms at positions corresponding to positions of PQRST complexes in the patient ECG signal. The impedance analysis system may process the one or more impedance waveforms to measure one or more parameters of the one or more impedance waveforms. The one or more parameters of the one or more impedance waveforms may comprise an amplitude of a peak of an impedance waveform, a width of a peak of an impedance waveform. The impedance analysis system may use the one or more parameters of the one or more impedance waveforms to generate a first impedance post-shock refibrillation indicator.

After delivery of a defibrillation shock, the impedance analysis system may analyse the patient impedance signal to detect one or more impedance waveforms indicating one or more breaths of the patient. The impedance analysis system may analyse the patient impedance signals to detect the one or more impedance waveforms indicating one or more breaths of the patient by using an artificial intelligence or a machine learning technique, which teaches the defibrillator to identify impedance waveforms that indicate one or more patient breaths. The impedance analysis system may process the one or more impedance waveforms to measure one or more parameters of the one or more impedance waveforms. The impedance analysis system may use the one or more parameters of the one or more impedance waveforms to generate a second impedance post-shock refibrillation indicator.

The refibrillation indicator model may combine the aggregate ECG refibrillation indicator and the first impedance post-shock refibrillation indicator and/or the second impedance post-shock refibrillation indicator to generate an aggregate ECG/impedance refibrillation indicator. The refibrillation indicator model may compare the aggregate ECG/impedance refibrillation indicator with an aggregate ECG/impedance refibrillation threshold and generate a flag which prompts the defibrillator to deliver a treatment to the patient. When the aggregate ECG/impedance refibrillation indicator is less than or equal to the aggregate ECG/impedance refibrillation threshold, the refibrillation indicator model may generate a flag which prompts the defibrillator to commence CPR. The defibrillator may issue a commence CPR signal. When the aggregate ECG/impedance refibrillation indicator is greater than the aggregate ECG/impedance refibrillation threshold, the refibrillation indicator model may generate a flag which prompts the defibrillator to delay CPR. The defibrillator may issue a delay CPR signal.

The refibrillation indicator model may weight the aggregate ECG refibrillation indicator and the first and second impedance refibrillation indicators. The refibrillation indicator model may combine the weighted aggregate ECG refibrillation indicator and the weighted first impedance post-shock refibrillation indicator and/or the weighted second impedance post-shock refibrillation indicator to generate an aggregate ECG/impedance refibrillation indicator.

The refibrillation indicator model may compare the aggregate ECG/impedance refibrillation indicator with an aggregate ECG/impedance refibrillation threshold and generate a flag which prompts the defibrillator to deliver a treatment to the patient. When the aggregate ECG/impedance refibrillation indicator is less than or equal to the aggregate ECG/impedance refibrillation threshold, the refibrillation indicator model may generate a flag which prompts the defibrillator to commence CPR. The defibrillator may issue a commence CPR signal. When the aggregate ECG/impedance refibrillation indicator is greater than the aggregate ECG/impedance refibrillation threshold, the refibrillation indicator model may generate a flag which prompts the defibrillator to delay CPR. The defibrillator may issue a delay CPR signal.

The aggregate ECG/impedance refibrillation threshold may be a predetermined threshold generated using data from patients who have received defibrillation shock therapy. The aggregate ECG/impedance refibrillation threshold may be a patient-based threshold generated using treatment history data of the patient.

The user interface may comprise an audio communication unit, such as a speaker, configured to receive the start CPR signal and deliver an audible start CPR instruction to the user and to receive the delay CPR signal and deliver an audible delay CPR instruction to the user. Additionally or alternatively, the user interface may comprise a visual communication unit, such as a display, configured to receive the start CPR signal and deliver a visual start CPR instruction to the user and to receive the delay CPR signal and deliver a visual delay CPR instruction to the user.

The defibrillator may comprise a defibrillation shock circuit configured to generate and deliver one or more defibrillation shocks to the patient.

The defibrillator may comprise a plurality of ECG electrodes adapted for attachment to the patient. The ECG electrodes may measure an ECG signal of the patient. The ECG electrodes may be in communication with the ECG signal input for the input to receive the ECG signal of the patient.

The defibrillator may comprise a plurality of impedance electrodes adapted for attachment to the patient. The impedance electrodes may measure an impedance signal of the patient. The impedance electrodes may be in communication with the impedance signal input for the input to receive the impedance signal of the patient.

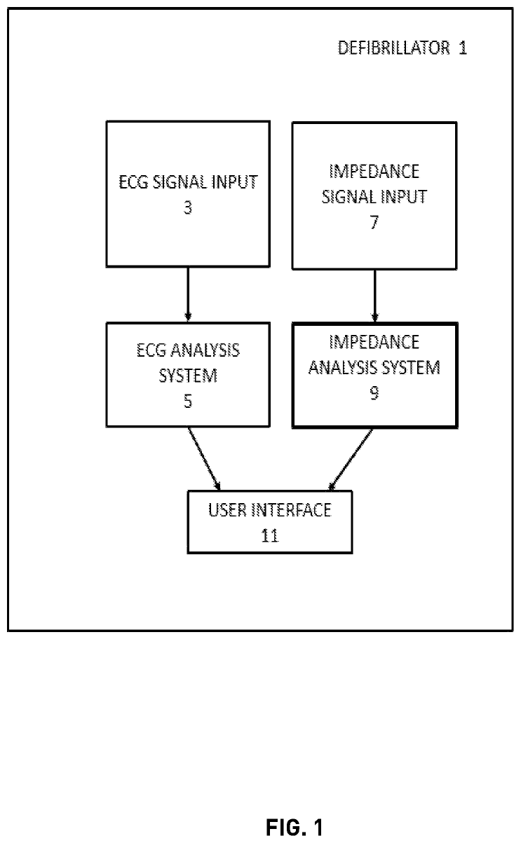

Referring to FIG. 1, a defibrillator 1 for assessing patient refibrillation, comprises an ECG signal input 3, configured to receive ECG signals of the patient, an ECG analysis system 5, in communication with the ECG signal input 3, an impedance signal input 7, configured to receive impedance signals of the patient, an impedance analysis system 9, in communication with the impedance signal input 7 and a user interface 11.

The defibrillator 1 comprises a plurality of ECG electrodes (not shown) adapted for attachment to the patient. The ECG electrodes measure an ECG signal of the patient and are in communication with the ECG signal input 3 for the input to receive the ECG signal of the patient. The defibrillator 1 further comprises a plurality of impedance electrodes (not shown) adapted for attachment to the patient. The impedance electrodes measure an impedance signal of the patient and are in communication with the impedance signal input 7 for the input to receive the impedance signal of the patient.

The ECG analysis system 5 and the impedance analysis system 9 comprise one or more processors (not shown), or other suitable hardware, to run one or more software algorithms for the analysis of the patient ECG signals and patient impedance signals.

The defibrillator 1 comprises a defibrillation shock circuit (not shown) configured to generate and deliver one or more defibrillation shocks to the patient.

It will be appreciated that the defibrillator 1 comprises other components, such as a power supply, which are not illustrated in FIG. 1.

Generally, the defibrillator 1 operates to receive the patient ECG signals, analyse the patient ECG signals to generate an ECG refibrillation indicator representing a likelihood of refibrillation of the patient and use the ECG refibrillation indicator to choose treatment for the patient. The defibrillator further operates to receive the patient impedance signals, analyse the patient impedance signals to generate an impedance refibrillation indicator representing a likelihood of refibrillation of the patient and use the impedance refibrillation indicator to choose treatment for the patient.

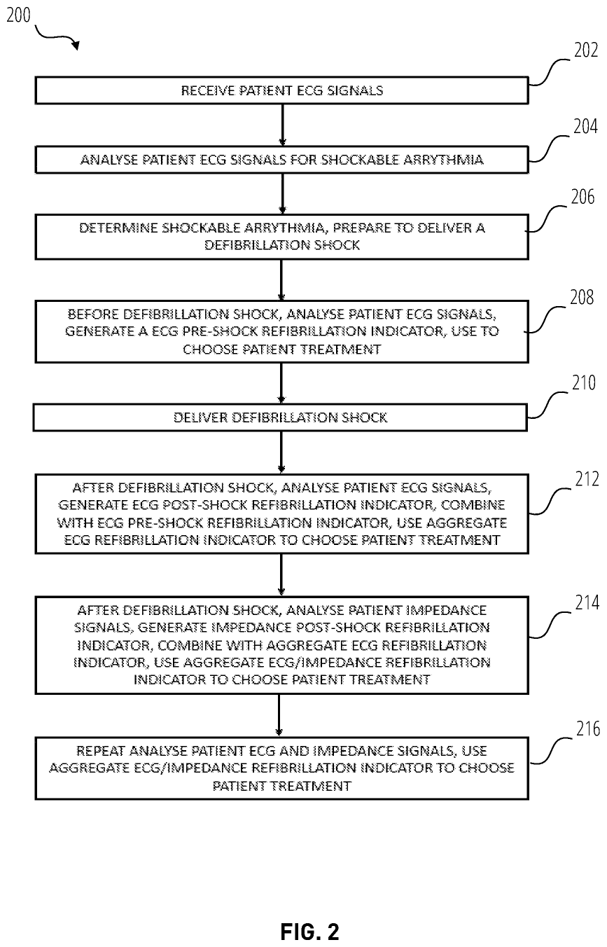

Referring to FIG. 2, an example process 200 carried out by the defibrillator 1 will be described. After application of the ECG electrodes to the patient, the defibrillator 1 the ECG signal input 3 receives patient ECG signals from the ECG electrodes (202). The defibrillator uses at least one algorithm to analyse the received ECG signals for a shockable arrythmia (204). The algorithm may be a set of software instructions run by the ECG analysis system 5 or another analysis component of the defibrillator 1. When a shockable arrythmia is determined, such as VF, the defibrillator 1 begins preparations to deliver a defibrillation shock (206).

The ECG analysis system 5 analyses the patient ECG signals before a defibrillation shock, generates a ECG pre-shock refibrillation indicator and uses this to choose treatment for the patient (208). Specifically, in one embodiment, the ECG analysis system 5 uses the ECG pre-shock refibrillation indicator to choose to deliver defibrillation shock treatment (210) to the patient at an energy equal to a default energy or deliver defibrillation shock treatment to the patient at an energy greater than the default energy.

The ECG analysis system 5 also analyses the patient ECG signals after a defibrillation shock and generates at least one ECG post-shock refibrillation indicator. This is combined with the ECG pre-shock refibrillation indicator to generate an aggregate ECG refibrillation indicator, which is used to choose treatment for the patient (212). Specifically, in one embodiment, the ECG analysis system 5 uses the aggregate ECG refibrillation indicator to choose to start CPR treatment of the patient or delay CPR treatment of the patient.

In this embodiment, the impedance analysis system 9 analyses the patient impedance signals after delivery of a defibrillation shock and generates at least one impedance refibrillation indicator. This is combined with the aggregate ECG refibrillation indicator to generate an aggregate ECG/impedance refibrillation indicator, which is used to choose treatment for the patient (214). Specifically, in one embodiment, the impedance analysis system 9 uses the aggregate ECG/impedance refibrillation indicator to choose to start CPR treatment of the patient or delay CPR treatment of the patient.

The ECG analysis system 5 and the impedance analysis system 9 issue a start CPR signal or issue a delay CPR signal, which is received by the user interface 11 which delivers a start CPR instruction to a user of the defibrillator 1 or a delay CPR instruction to the user. The user interface 11 comprises an audio communication unit, such as a speaker, configured to receive the start CPR signal and deliver an audible start CPR instruction to the user and to receive the delay CPR signal and deliver an audible delay CPR instruction to the user. Additionally or alternatively, the user interface 11 comprises a visual communication unit, such as a display, configured to receive the start CPR signal and deliver a visual start CPR instruction to the user and to receive the delay CPR signal and deliver a visual delay CPR instruction to the user.

Repeated analysis of the patient ECG and impedance signals is carried out and further aggregate ECG/impedance refibrillation indicators are generated and used to choose treatment for the patient (216).

When patient treatment comprising CPR delay is chosen, repeated analysis of the patient ECG signals is carried out. If during the delay, a shockable patient heart arrythmia is detected, the defibrillator 1 prepares for and delivers a defibrillation shock.

It will be appreciated that, in other embodiments, analysis of patient impedance signals may be omitted and the patient treatment chosen using ECG refibrillation indicators only.

In more detail, before delivery of a defibrillation shock, the ECG analysis system 5 takes one or more samples of the ECG signals over one or more periods of time. The or each period of time may be in the range of 1 sec to 10 sec. When two or more samples are taken, the periods of time may be any of adjacent periods of time, spaced periods of time.

In this embodiment, the ECG analysis system 5 analyses one or more whole samples of the ECG signals to detect one or more parameters of the or each whole sample. The ECG analysis system 5 then uses the or each or at least some of the parameters and/or aggregated parameters as one or more inputs of a refibrillation indicator model which generates an ECG pre-shock refibrillation indicator.

Alternatively, the ECG analysis system 5 may analyse one or more samples of the ECG signals to detect one or more parameters at one or more sample instances of the or each sample and use the or each or at least some of the parameters and/or aggregated parameters as one or more inputs of a refibrillation indicator model which generates an ECG pre-shock refibrillation indicator.

Before delivery of a defibrillation shock, it is expected that the ECG analysis system 5 receives ECG signals having an arrythmia waveform, e.g. a VF waveform. The one or more parameters will then be parameters of the VF waveform.

The one or more parameters may be any of time domain parameters, frequency domain parameters, wavelet parameters. The one or more parameters may be any of an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum amplitude spectrum area (AMSA), an ECG signal sample minimum AMSA, an ECG signal sample centroid frequency, an ECG signal sample power.

The refibrillation indicator model may weight each input according to its strength of indication of refibrillation. The strengths of indication of refibrillation of the inputs may be determined from research. The refibrillation indicator model may use the weighted inputs to generate an ECG pre-shock refibrillation indicator.

For multiple samples, the refibrillation indicator model may generate an ECG pre-shock refibrillation indicator for each sample and aggregate the ECG pre-shock refibrillation indicators to generate an ECG pre-shock refibrillation indicator. For multiple samples, the refibrillation indicator model may weight the ECG pre-shock refibrillation indicators and then aggregate the weighted ECG pre-shock refibrillation indicators to generate an ECG pre-shock refibrillation indicator.

The refibrillation indicator model may be a binary logistic regression model. The regression model may determine a likelihood of refibrillation using the one or more inputs of the model.

The refibrillation indicator model compares the ECG pre-shock refibrillation indicator to at least one pre-determined ECG pre-shock refibrillation threshold and generates a flag which prompts the defibrillator to deliver a treatment to the patient. The at least one pre-determined ECG pre-shock refibrillation threshold may be generated using data from patients who have received defibrillation shock therapy and/or using data from the patient receiving defibrillation shock therapy. When the ECG pre-shock refibrillation indicator is less than or equal to the pre-determined ECG pre-shock refibrillation threshold, the refibrillation indicator model generates a flag which prompts the defibrillator 1 to deliver defibrillation shock treatment to the patient at an energy equal to a default energy. When the ECG pre-shock refibrillation indicator is greater than the pre-determined ECG pre-shock refibrillation threshold, the refibrillation indicator model generates a flag which prompts the defibrillator 1 to deliver defibrillation shock treatment to the patient at an energy greater than the default energy.

After delivery of the defibrillation shock, the ECG analysis system 5 take one or more samples of the ECG signals over one or more periods of time. In this embodiment, the ECG analysis system 5 analyse one or more whole samples of the ECG signal to detect one or more parameters of the or each whole sample and uses the or each or at least some of the parameters and/or aggregated parameters as one or more inputs of a refibrillation indicator model which generates an ECG post-shock refibrillation indicator.

In other embodiments, the ECG analysis system 5 may, additionally or alternatively, analyse one or more samples of the ECG signal to detect one or more parameters at one or more sample instances of the or each sample and use the or each or at least some of the parameters and/or aggregated parameters as one or more inputs of a refibrillation indicator model which generates an ECG post-shock refibrillation indicator.

The one or more parameters may be any of time domain parameters, frequency domain parameters, wavelet parameters. The one or more parameters may be any of an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum AMSA, an ECG signal sample minimum AMSA, an ECG signal sample centroid frequency, an ECG signal sample power.

After delivery of a defibrillation shock, it is expected that the ECG analysis system 5 receives ECG signals having a waveform exhibiting one or more PQRST complexes. The one or more parameters may comprise any of an amplitude of a P wave of a PQRST complex, an amplitude of a QRS wave of a PQRST complex, variation of amplitudes of QRS waves of two or more PQRST complexes, an amplitude of a T wave of a PQRST complex, a width of a P wave of a PQRST complex, a width of a QRS wave of a PQRST complex, a width of a T wave of a PQRST complex, a ST segment of a PQRST complex, a coupling interval between a QRS complex and a premature, abnormal complex, an R-R interval between two or more PQRST complexes, a ST deviation, presence of one or more premature ventricular contractions.

The refibrillation indicator model may weight each input according to its strength of indication of refibrillation. The strengths of indication of refibrillation of the inputs may be determined from research. The refibrillation indicator model may use the weighted inputs to generate an ECG post-shock refibrillation indicator.

For multiple samples, the refibrillation indicator model may generate an ECG post-shock refibrillation indicator for each sample. For multiple samples, the refibrillation indicator model may aggregate the ECG post-shock refibrillation indicators to generate an ECG post-shock refibrillation indicator. For multiple samples, the refibrillation indicator model may weight the ECG post-shock refibrillation indicators and then aggregate the weighted ECG post-shock refibrillation indicators to generate an ECG post-shock refibrillation indicator.

The refibrillation indicator model may be a logistic regression model. The logistical regression model may determine a likelihood of refibrillation using the one or more inputs of the model.

The refibrillation indicator model combines the ECG pre-shock refibrillation indicator and the ECG post-shock refibrillation indicator to generate an aggregate ECG refibrillation indicator. The refibrillation indicator model may weight the ECG pre-shock refibrillation indicator, weight the ECG post-shock refibrillation indicator and combine the weighted ECG pre-shock refibrillation indicator and the weighted ECG post-shock refibrillation indicator to generate an aggregate ECG refibrillation indicator.

The refibrillation indicator model compares the aggregate ECG refibrillation indicator with at least one pre-determined aggregate ECG refibrillation threshold and generates a flag which prompts the defibrillator to deliver a treatment to the patient. The at least one pre-determined aggregate ECG refibrillation threshold may be generated using data from patients who have received defibrillation shock therapy and/or using data from the patient receiving defibrillation shock therapy. When the aggregate ECG refibrillation indicator is less than or equal to the at least one pre-determined ECG refibrillation threshold, the refibrillation indicator model generates a flag which prompts the defibrillator 1 to start CPR treatment of the patient and to issue a start CPR signal. The user interface 11 of the defibrillator 1 receives the start CPR signal and delivers a start CPR instruction to the user. When the aggregate ECG refibrillation indicator is greater than the at least one pre-determined ECG refibrillation threshold, the refibrillation indicator model generates a flag which prompts the defibrillator 1 to delay CPR treatment of the patient and to issue a delay CPR signal. The user interface 11 of the defibrillator 1 receives the delay CPR signal and delivers a delay CPR instruction to the user.

At one or more further times after delivery of the defibrillation shock, the ECG analysis system repeats analysis of the patient ECG signals and the refibrillation indicator model generates a further ECG post-shock refibrillation indicator. The refibrillation indicator model combines the ECG pre-shock refibrillation indicator and the further ECG post-shock refibrillation indicator to generate an aggregate ECG refibrillation indicator.

The refibrillation indicator model compares the aggregate ECG refibrillation indicator with at least one pre-determined aggregate ECG refibrillation threshold and generates a flag which prompts the defibrillator to deliver a treatment to the patient. When the aggregate ECG refibrillation indicator is less than or equal to the at least one pre-determined ECG refibrillation threshold, the refibrillation indicator model again generates a flag which prompts the defibrillator 1 to start CPR treatment of the patient and to issue a start CPR signal. When the aggregate ECG refibrillation indicator is greater than the at least one pre-determined ECG refibrillation threshold, the refibrillation indicator model generates a flag which prompts the defibrillator 1 to delay CPR treatment of the patient and to issue a delay CPR signal.

In this embodiment, after delivery of the defibrillation shock, the impedance analysis system 9 analyses the patient impedance signals to detect one or more impedance waveforms indicating one or more contractions of the patient's heart, by searching for the one or more impedance waveforms at positions corresponding to positions of PQRST complexes in the patient ECG signals. The impedance analysis system 9 processes the one or more impedance waveforms to measure one or more parameters. The one or more parameters may comprise an amplitude of a peak of an impedance waveform, a width of a peak of an impedance waveform. The impedance analysis system 9 uses the one or more parameters of the one or more impedance waveforms to generate a first impedance post-shock refibrillation indicator.

The impedance analysis system 9 further analyses the patient impedance signal to detect one or more impedance waveforms indicating one or more breaths of the patient by using an artificial intelligence or a machine learning technique, which teaches the defibrillator to identify impedance waveforms that indicate one or more patient breaths. The impedance analysis system 9 processes the one or more impedance waveforms to measure one or more parameters and uses the one or more parameters to generate a second impedance post-shock refibrillation indicator.

The refibrillation indicator model combines the aggregate ECG refibrillation indicator and the first impedance post-shock refibrillation indicator and the second impedance post-shock refibrillation indicator to generate an aggregate ECG/impedance refibrillation indicator. The refibrillation indicator model compares the aggregate ECG/impedance refibrillation indicator to an aggregate ECG/impedance refibrillation threshold.

The refibrillation indicator model may weight the aggregate ECG refibrillation indicator and the first and second impedance refibrillation indicators. The refibrillation indicator model may combine the weighted aggregate ECG refibrillation indicator and the weighted first impedance post-shock refibrillation indicator and the weighted second impedance post-shock refibrillation indicator to generate an aggregate ECG/impedance refibrillation indicator.

The refibrillation indicator model compares the aggregate ECG/impedance refibrillation indicator with an aggregate ECG/impedance refibrillation threshold. When the aggregate ECG/impedance refibrillation indicator is less than or equal to the aggregate ECG/impedance refibrillation threshold, the refibrillation indicator model prompts the defibrillator 1 to issue a commence CPR signal. When the aggregate ECG/impedance refibrillation indicator is greater than the aggregate ECG/impedance refibrillation threshold, the refibrillation indicator model prompts the defibrillator 1 to issue a delay CPR signal.

During any CPR delay time, the ECG analysis system 5 receives and analyses patient ECG signals. If a shockable arrythmia is determined, the defibrillator 1 delivers a further defibrillation shock.

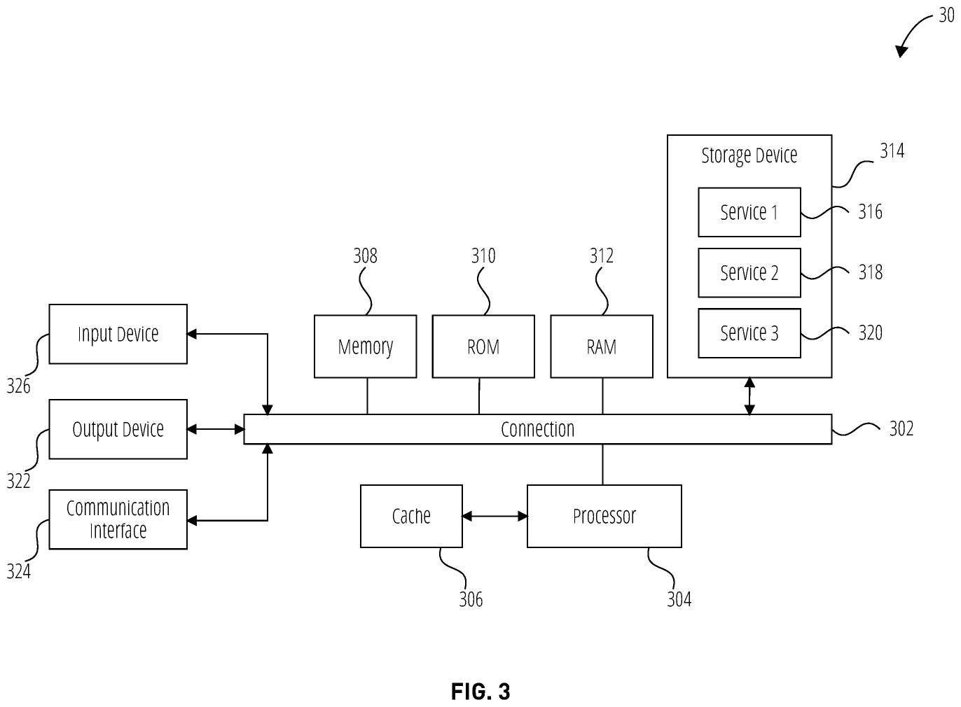

FIG. 3 shows an example of computing system 300, which can be for example any computing device making up the defibrillator 1, or any component thereof including components of the system that are in communication with each other using connection 302. Connection 302 can be a physical connection via a bus, or a direct connection into processor 304, such as in a chipset architecture. Connection 302 can also be a virtual connection, networked connection, or logical connection.

In some embodiments, computing system 300 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example computing system 300 includes at least one processing unit (CPU or processor) 1004 and connection 302 that couples various system components including system memory 308, such as read-only memory (ROM) 1010 and random access memory (RAM) 1012 to processor 1004. Computing system 1000 can include a cache of high-speed memory 1006 connected directly with, in close proximity to, or integrated as part of processor 1004.

Processor 1004 can include any general purpose processor and a hardware service or software service, such as services 1016, 1018, and 1020 stored in storage device 1014, configured to control processor 1004 as well as a special-purpose processor where software instructions are incorporated into the actual processor design. Processor 1004 may essentially be a completely self-contained computing system, containing multiple cores or processors, a bus, memory controller, cache, etc. A multi-core processor may be symmetric or asymmetric.

To enable user interaction, computing system 1000 includes an input device 1026, which can represent any number of input mechanisms, such as a microphone for speech, a touch-sensitive screen for gesture or graphical input, keyboard, mouse, motion input, speech, etc. Computing system 1000 can also include output device 1022, which can be one or more of a number of output mechanisms known to those of skill in the art. In some instances, multimodal systems can enable a user to provide multiple types of input/output to communicate with computing system 100. Computing system 1000 can include communication interface 1024, which can generally govern and manage the user input and system output. There is no restriction on operating on any particular hardware arrangement, and therefore the basic features here may easily be substituted for improved hardware or firmware arrangements as they are developed.

Storage device 1014 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 1014 can include software services, servers, services, etc., that when the code that defines such software is executed by the processor 1004, it causes the system to perform a function. In some embodiments, a hardware service that performs a particular function can include the software component stored in a computer-readable medium in connection with the necessary hardware components, such as processor 1004, connection 1002, output device 1022, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Claim clauses include the following:

Clause 1. A defibrillator for assessing a likelihood of patient refibrillation, comprising: an electrocardiogram (ECG) signal input configured to receive ECG signals of a patient; and an ECG analysis system, in communication with the ECG signal input, configured to receive patient ECG signals, analyse the patient ECG signals to generate an ECG refibrillation indicator representing a likelihood of refibrillation of the patient and use the ECG refibrillation indicator to choose treatment for the patient.

Clause 2. The defibrillator of clause 1, wherein the ECG analysis system analyses the patient ECG signals before a defibrillation shock and generates an ECG pre-shock refibrillation indicator and the defibrillator uses the ECG pre-shock refibrillation indicator to choose treatment for the patient.

Clause 3. A defibrillator according to clause 2, wherein the defibrillator uses the ECG pre-shock refibrillation indicator to choose to deliver defibrillation shock treatment to the patient at an energy equal to a default energy or deliver defibrillation shock treatment to the patient at an energy greater than the default energy.

Clause 4. A defibrillator according to clause 2 or claim 3, wherein the ECG analysis system analyses the patient ECG signals after delivery of a defibrillation shock and generates at least one ECG post-shock refibrillation indicator and the defibrillator combines the ECG pre-shock refibrillation indicator and the at least one ECG post-shock refibrillation indicator to generate an aggregate ECG refibrillation indicator and uses the aggregate ECG refibrillation indicator to choose treatment for the patient.

Clause 5. A defibrillator according to clause 4, wherein the defibrillator uses the aggregate ECG refibrillation indicator to choose to start a cardiopulmonary resuscitation (CPR) treatment of the patient or delay the CPR treatment of the patient.

Clause 6. A defibrillator according to any preceding clause in which, before delivery of a defibrillation shock, the ECG analysis system takes one or more samples of the ECG signals over one or more periods of time, analyses the one or more samples of the ECG signals to detect one or more parameters of the one or more samples of the ECG signals and uses at least some of the one or more parameters as one or more inputs of a refibrillation indicator model which generates an ECG pre-shock refibrillation indicator.

Clause 7. A defibrillator according to clause 6, wherein the one or more parameters are any of time domain parameters, frequency domain parameters, wavelet parameters, an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum amplitude spectrum area (AMSA), an ECG signal sample minimum AMSA, an ECG signal sample centroid frequency, an ECG signal sample power, parameters of a VF waveform.

Clause 8. A defibrillator according to clause 6 or claim 7, wherein the refibrillation indicator model compares the ECG pre-shock refibrillation indicator to at least one pre-determined ECG pre-shock refibrillation threshold and generates a flag which prompts the defibrillator to deliver a treatment to the patient.

Clause 9. A defibrillator according to any preceding clause, wherein after delivery of a defibrillation shock, the ECG analysis system takes one or more samples of the ECG signals over one or more periods of time, analyses one or more samples of the ECG signals to detect one or more parameters of the one or more samples of the ECG signals and uses at least some of the one or more parameters as one or more inputs of a refibrillation indicator model which generates an ECG post-shock refibrillation indicator.

Clause 10. A defibrillator according to clause 9, wherein the one or more parameters are any of time domain parameters, frequency domain parameters, wavelet parameters, an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum AMSA, an ECG signal sample minimum AMSA, an ECG signal sample centroid frequency, an ECG signal sample power, an amplitude of a P wave of a PQRST (provocation, quality, region, severity and timing) complex, an amplitude of a QRS wave of a PQRST complex, variation of amplitudes of QRS waves of two or more PQRST complexes, an amplitude of a T wave of a PQRST complex, a width of a P wave of a PQRST complex, a width of a QRS wave of a PQRST complex, a width of a T wave of a PQRST complex, a ST segment of a PQRST complex, a coupling interval between a QRS complex and a premature, abnormal complex, an R-R interval between two or more PQRST complexes, a ST deviation, presence of one or more premature ventricular contractions.

Clause 11. A defibrillator according to clause 9 or claim 10, wherein the refibrillation indicator model combines the ECG pre-shock refibrillation indicator and the ECG post-shock refibrillation indicator to generate an aggregate ECG refibrillation indicator, compares the aggregate ECG refibrillation indicator with at least one pre-determined aggregate ECG refibrillation threshold and generates a flag which prompts the defibrillator to deliver a treatment to the patient.

Clause 12. A defibrillator according to clause 11, wherein, when the aggregate ECG refibrillation indicator is less than or equal to the at least one pre-determined ECG refibrillation threshold, the refibrillation indicator model generates a flag which prompts the defibrillator to start CPR treatment of the patient and, when the aggregate ECG refibrillation indicator is greater than the at least one pre-determined ECG refibrillation threshold, the refibrillation indicator model generates a flag which prompts the defibrillator (1) to delay CPR treatment of the patient.

Clause 13. A defibrillator according to any of clauses 9 to 12, wherein at one or more further times after delivery of the defibrillation shock, the ECG analysis system (5) repeats analysis of the patient ECG signal, the refibrillation indicator model generates a further ECG post-shock refibrillation indicator and combines the ECG pre-shock refibrillation indicator and the further ECG post-shock refibrillation indicator to generate an aggregate ECG refibrillation indicator, compares the aggregate ECG refibrillation indicator with at least one pre-determined aggregate ECG refibrillation threshold and generates a flag which prompts the defibrillator to deliver a treatment to the patient.

Clause 14. A defibrillator according to any preceding clause, the defibrillator further comprising: an impedance signal input configured to receive impedance signals of the patient and an impedance analysis system, in communication with the impedance signal input, configured to receive the patient impedance signal, analyse the patient impedance signal to generate at least one impedance refibrillation indicator representing a likelihood of refibrillation of the patient and the defibrillator uses the at least one impedance refibrillation indicator to choose treatment for the patient.

Clause 15. A defibrillator according to clause 14, as dependent from claim 11, wherein the refibrillation indicator model combines the aggregate ECG refibrillation indicator and the at least one impedance refibrillation indicator to generate an aggregate ECG/impedance refibrillation indicator, compares the aggregate ECG/impedance refibrillation indicator with an aggregate ECG/impedance refibrillation threshold and generates a flag which prompts the defibrillator to deliver a treatment to the patient.

Clause 16. A method comprising: receiving patient ECG signals; analyzing the patient ECG signals for shockable arrythmia; determining the shockable arrythmia and preparing to deliver a defibrillation shock; before the defibrillation shock, analyzing the patient ECG signals and generating an ECG pre-shock refibrillation indicator used to choose a patient treatment; delivering the defibrillation shock; after the defibrillation shock, analysing the patient ECG signals and generating an ECG post-shock refibrillation indicator, combining the ECG post-shock refibrillation indicator with an ECG pre-shock refibrillation indicator to generate an aggregate ECG refibrillation indicator; using the aggregate ECG refibrillation indicator to choose the patient treatment; after the defibrillation shock, analysing patient impedance signals, generating an impedance post-shock refibrillation indicator, combing the impedance post-shock refibrillation indicator with the aggregate ECG refibrillation indicator to generate an aggregate ECG/impedance refibrillation indicator; using the aggregate ECG/impedance refibrillation indicator to choose the patient treatment; and repeating the analyzing of the patient ECG signals and the analyzing of the patient impedance signals and using an updated aggregated ECG/impedance refibrillation indicator to choose the patient treatment.

Clause 17. A device comprising: a processor; a computer-readable memory storing instructions which, when executed by the processor, cause the processor to perform operations comprising: receiving patient ECG signals; analyzing the patient ECG signals for shockable arrythmia; determining the shockable arrythmia and preparing to deliver a defibrillation shock; before the defibrillation shock, analyzing the patient ECG signals and generating an ECG pre-shock refibrillation indicator used to choose a patient treatment; delivering the defibrillation shock; after the defibrillation shock, analysing the patient ECG signals and generating an ECG post-shock refibrillation indicator, combining the ECG post-shock refibrillation indicator with an ECG pre-shock refibrillation indicator to generate an aggregate ECG refibrillation indicator; using the aggregate ECG refibrillation indicator to choose the patient treatment; after the defibrillation shock, analysing patient impedance signals, generating an impedance post-shock refibrillation indicator, combing the impedance post-shock refibrillation indicator with the aggregate ECG refibrillation indicator to generate an aggregate ECG/impedance refibrillation indicator; using the aggregate ECG/impedance refibrillation indicator to choose the patient treatment; and repeating the analyzing of the patient ECG signals and the analyzing of the patient impedance signals and using an updated aggregated ECG/impedance refibrillation indicator to choose the patient treatment.

What is claimed is:

1. A defibrillator for assessing a likelihood of patient refibrillation, comprising:
an electrocardiogram (ECG) signal input configured to receive ECG signals of a patient; and
an ECG analysis system, in communication with the ECG signal input, configured to receive patient ECG signals, analyse the patient ECG signals to generate an ECG refibrillation indicator representing a likelihood of refibrillation of the patient and use the ECG refibrillation indicator to choose treatment for the patient.

2. The defibrillator of claim 1, wherein the ECG analysis system analyses the patient ECG signals before a defibrillation shock and generates an ECG pre-shock refibrillation indicator and the defibrillator uses the ECG pre-shock refibrillation indicator to choose treatment for the patient.

3. A defibrillator of claim 2, wherein the defibrillator uses the ECG pre-shock refibrillation indicator to choose to deliver defibrillation shock treatment to the patient at an energy equal to a default energy or deliver defibrillation shock treatment to the patient at an energy greater than the default energy.

4. A defibrillator of claim 2, wherein the ECG analysis system analyses the patient ECG signals after delivery of a defibrillation shock and generates at least one ECG post-shock refibrillation indicator and the defibrillator combines the ECG pre-shock refibrillation indicator and the at least one ECG post-shock refibrillation indicator to generate an aggregate ECG refibrillation indicator and uses the aggregate ECG refibrillation indicator to choose treatment for the patient.

5. A defibrillator according to claim 4, wherein the defibrillator uses the aggregate ECG refibrillation indicator to choose to start a cardiopulmonary resuscitation (CPR) treatment of the patient or delay the CPR treatment of the patient.

6. A defibrillator of claim 1, wherein, before delivery of a defibrillation shock, the ECG analysis system takes one or more samples of the ECG signals over one or more periods of time, analyses the one or more samples of the ECG signals to detect one or more parameters of the one or more samples of the ECG signals and uses at least some of the one or more parameters as one or more inputs of a refibrillation indicator model which generates an ECG pre-shock refibrillation indicator.

7. A defibrillator according to claim 6, wherein the one or more parameters are any of time domain parameters, frequency domain parameters, wavelet parameters, an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum amplitude spectrum area (AMSA), an ECG signal sample minimum AMSA, an ECG signal sample centroid frequency, an ECG signal sample power, parameters of a VF waveform.

8. A defibrillator of claim 6, wherein the refibrillation indicator model compares the ECG pre-shock refibrillation indicator to at least one pre-determined ECG pre-shock refibrillation threshold and generates a flag which prompts the defibrillator to deliver a treatment to the patient.

9. A defibrillator of claim 8, wherein after delivery of a defibrillation shock, the ECG analysis system takes one or more samples of the ECG signals over one or more periods of time, analyses one or more samples of the ECG signals to detect one or more parameters of the one or more samples of the ECG signals and uses at least some of the one or more parameters as one or more inputs of a refibrillation indicator model which generates an ECG post-shock refibrillation indicator.

10. A defibrillator of claim 9, wherein the one or more parameters are any of time domain parameters, frequency domain parameters, wavelet parameters, an ECG signal sample maximum amplitude, an ECG signal sample minimum amplitude, an ECG signal sample maximum positive slope, an ECG signal sample minimum positive slope, an ECG signal sample maximum negative slope, an ECG signal sample minimum negative slope, an ECG signal sample maximum AMSA, an ECG signal sample minimum AMSA, an ECG signal sample centroid frequency, an ECG signal sample power, an amplitude of a P wave of a PQRST (provocation, quality, region, severity and timing) complex, an amplitude of a QRS wave of a PQRST complex, variation of amplitudes of QRS waves of two or more PQRST complexes, an amplitude of a T wave of a PQRST complex, a width of a P wave of a PQRST complex, a width of a QRS wave of a PQRST complex, a width of a T wave of a PQRST complex, a ST segment of a PQRST complex, a coupling interval between a QRS complex and a premature, abnormal complex, an R-R interval between two or more PQRST complexes, a ST deviation, presence of one or more premature ventricular contractions.

11. A defibrillator of claim 9, wherein the refibrillation indicator model combines the ECG pre-shock refibrillation indicator and the ECG post-shock refibrillation indicator to generate an aggregate ECG refibrillation indicator, compares the aggregate ECG refibrillation indicator with at least one pre-determined aggregate ECG refibrillation threshold and generates a flag which prompts the defibrillator to deliver a treatment to the patient.

12. A defibrillator of claim 11, wherein, when the aggregate ECG refibrillation indicator is less than or equal to the at least one pre-determined ECG refibrillation threshold, the refibrillation indicator model generates a flag which prompts the defibrillator to start CPR treatment of the patient and, when the aggregate ECG refibrillation indicator is greater than the at least one pre-determined ECG refibrillation threshold, the refibrillation indicator model generates a flag which prompts the defibrillator (1) to delay CPR treatment of the patient.

13. A defibrillator of claim 12, wherein at one or more further times after delivery of the defibrillation shock, the ECG analysis system (5) repeats analysis of the patient ECG signal, the refibrillation indicator model generates a further ECG post-shock refibrillation indicator and combines the ECG pre-shock refibrillation indicator and the further ECG post-shock refibrillation indicator to generate an aggregate ECG refibrillation indicator, compares the aggregate ECG refibrillation indicator with at least one pre-determined aggregate ECG refibrillation threshold and generates a flag which prompts the defibrillator to deliver a treatment to the patient.

14. A defibrillator of claim 11, the defibrillator further comprising:
an impedance signal input configured to receive impedance signals of the patient and an impedance analysis system, in communication with the impedance signal input, configured to receive the patient impedance signal, analyse the patient impedance signal to generate at least one impedance refibrillation indicator representing a likelihood of refibrillation of the patient and the defibrillator uses the at least one impedance refibrillation indicator to choose treatment for the patient.

15. A defibrillator of claim 14, wherein the refibrillation indicator model combines the aggregate ECG refibrillation indicator and the at least one impedance refibrillation indicator to generate an aggregate ECG/impedance refibrillation indicator, compares the aggregate ECG/impedance refibrillation indicator with an aggregate ECG/impedance refibrillation threshold and generates a flag which prompts the defibrillator to deliver a treatment to the patient.

16. A defibrillator of claim 1, the defibrillator further comprising:
an impedance signal input configured to receive impedance signals of the patient and an impedance analysis system, in communication with the impedance signal input, configured to receive the patient impedance signal, analyse the patient impedance signal to generate at least one impedance refibrillation indicator representing a likelihood of refibrillation of the patient and the defibrillator uses the at least one impedance refibrillation indicator to choose treatment for the patient.

17. A defibrillator of claim 10, wherein the refibrillation indicator model combines the ECG pre-shock refibrillation indicator and the ECG post-shock refibrillation indicator to generate an aggregate ECG refibrillation indicator, compares the aggregate ECG refibrillation indicator with at least one pre-determined aggregate ECG refibrillation threshold and generates a flag which prompts the defibrillator to deliver a treatment to the patient.

18. A defibrillator of claim 7, wherein the refibrillation indicator model compares the ECG pre-shock refibrillation indicator to at least one pre-determined ECG pre-shock refibrillation threshold and generates a flag which prompts the defibrillator to deliver a treatment to the patient.

19. A method comprising:
receiving patient ECG signals;
analyzing the patient ECG signals for shockable arrythmia;
determining the shockable arrythmia and preparing to deliver a defibrillation shock;
before the defibrillation shock, analyzing the patient ECG signals and generating an ECG pre-shock refibrillation indicator used to choose a patient treatment;
delivering the defibrillation shock;
after the defibrillation shock, analysing the patient ECG signals and generating an ECG post-shock refibrillation indicator, combining the ECG post-shock refibrillation indicator with an ECG pre-shock refibrillation indicator to generate an aggregate ECG refibrillation indicator;
using the aggregate ECG refibrillation indicator to choose the patient treatment;
after the defibrillation shock, analysing patient impedance signals, generating an impedance post-shock refibrillation indicator, combing the impedance post-shock refibrillation indicator with the aggregate ECG refibrillation indicator to generate an aggregate ECG/impedance refibrillation indicator;
using the aggregate ECG/impedance refibrillation indicator to choose the patient treatment; and
repeating the analyzing of the patient ECG signals and the analyzing of the patient impedance signals and using an updated aggregated ECG/impedance refibrillation indicator to choose the patient treatment.

20. A device comprising:
a processor;
a computer-readable memory storing instructions which, when executed by the processor, cause the processor to perform operations comprising:
receiving patient ECG signals;
analyzing the patient ECG signals for shockable arrythmia;
determining the shockable arrythmia and preparing to deliver a defibrillation shock;
before the defibrillation shock, analyzing the patient ECG signals and generating an ECG pre-shock refibrillation indicator used to choose a patient treatment;
delivering the defibrillation shock;
after the defibrillation shock, analysing the patient ECG signals and generating an ECG post-shock refibrillation indicator, combining the ECG post-shock refibrillation indicator with an ECG pre-shock refibrillation indicator to generate an aggregate ECG refibrillation indicator;
using the aggregate ECG refibrillation indicator to choose the patient treatment;
after the defibrillation shock, analysing patient impedance signals, generating an impedance post-shock refibrillation indicator, combing the impedance post-shock refibrillation indicator with the aggregate ECG refibrillation indicator to generate an aggregate ECG/impedance refibrillation indicator;
using the aggregate ECG/impedance refibrillation indicator to choose the patient treatment; and
repeating the analyzing of the patient ECG signals and the analyzing of the patient impedance signals and using an updated aggregated ECG/impedance refibrillation indicator to choose the patient treatment.
